(19) United States
(12) Plant Patent Application Publication
(10) Pub. No.: US 2004/0010828 P1
(43) Pub. Date: Jan. 15, 2004

(54) ILEX INTERSPECIFIC HYBRID NAMED 'RUTZAN'

(76) Inventors: Elwin R. Orton, Millstone, NJ (US); David A. Gant, Wall Township, NJ (US)

Correspondence Address: DRIGGS, LUCAS, BRUBAKER & HOGG CO., L.P.A., DEPT. DLBH, 8522 EAST AVENUE, MENTOR, OH, 44060, US

(21) Appl. No.: 10/195,001

(22) Filed: Jul. 11, 2002

(51) Int. Cl. A01H 5/00

(52) U.S. Cl. PLT/247

(57) ABSTRACT

Rutzan is a new and distinct variety of evergreen holly distinguished in that it combines from three species (Ilex aquifolium, Ilex rugosa, and Ilex pernyi), desirable landscape and production traits which distinguish it from all other forms of Ilex. Plants of the new variety exhibit a moderate rate of growth, develop a densely branched narrow to moderately broad conical form of compact size, possess small to medium sized dark green spiny leaves, produce an abundance of bright red fruit well displayed on wood of the previous season's growth, and are outstanding for their exceptionally high level of winter hardiness.

### BACKGROUND OF THE PRESENT INVENTION

Our breeding objective was to create an evergreen holly tree which embodies our ideas of desirability and other characteristics which would make the same commercially attractive and thereby having high potential for commercial distribution, and we believe we have been successful in achieving that goal. Other important selection factors may include ultimate plant size and shape, disease resistance, tolerance to different soil and growing conditions and vigor.

To our knowledge, plants of these three species combination have not been reported previously. The new clone has been named the Rutzan variety.

### BRIEF SUMMARY OF THE INVENTION

Our invention is a novel, unique, and highly ornamental form of Ilex which was originated by us by crossing an unnamed and unpatented F₂ generation pistillate seedling of Ilex×Meserveae (Ilex rugosa×Ilex aquifolium) with an unnamed and unpatented staminate seedling of the species known botanically as Ilex pernyi.

The variety exhibits the following combination of traits:

(a) a moderately vigorous, dense and self-compacting evergreen tree of a narrow to moderately broad conical form of much less height and size than that of most tree forms of Ilex aquifolium,

(b) evergreen leaves which are primarily ovate with a truncate base, spinose margin, slightly reflexed acuminate tip, and which are dark green and shiny,

(c) an abundance of pistillate flowers well positioned on shoots of the previous year's growth and providing a well distributed and very showy set of fruit maturing to a bright red in the early fall and then persisting throughout the winter,

(d) winter hardiness that we believe to be superior to that of all varieties of I. aquifolium and I. pernyi, and heat tolerance superior to that to all varieties of Ilex Aquifolium and Ilex rugosa, and

(e) low susceptibility to disease and insect pests.

In a production environment, plants of Rutzan variety develop a dense, self-compacting, conical growth habit with strong apical dominance without corrective pruning, and their small size relative to the larger tree form typical of plants of Ilex aquifolium, or hybrids of Ilex aquifolium×Ilex pernyi, makes them useful in a wider range of landscape settings.

### BRIEF DESCRIPTIONS OF THE DRAWINGS

The color definitions in the specification have been taken from the RHS Colour Chart of The Royal Horticultural Society, London, England. The colors depicted are believed to be of a high level of color fidelity and are believed to be as close to the actual coloration of the plant as possible in a photographic illustration of this quality. However, due to factors such as light reflectance, cultural conditions and horticultural practices, the coloration of this plant should be understood to be approximate. For example, the leaf color may vary depending on the composition and the concentration of fertilizer that may be applied to the plant.

FIG. 1 illustrates the natural unpruned growth habit and foliage and fruit characteristics of a nine year cutting-grown plant of the new variety, and

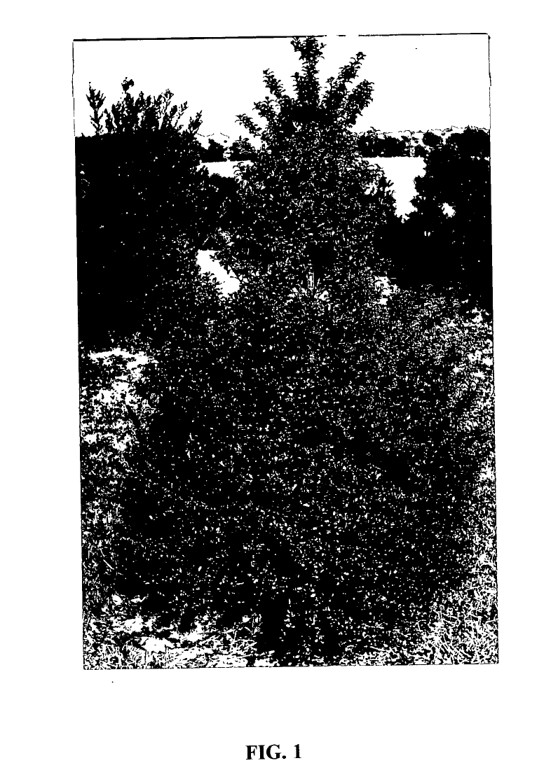

FIG. 2 illustrates typical foliage and fruit of the new variety.

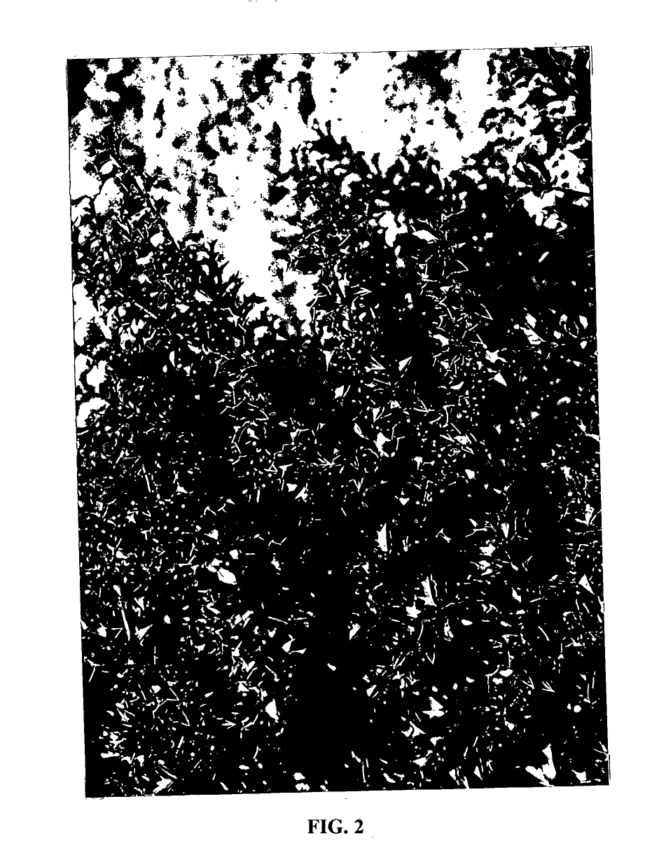

### BOTANICAL DESCRIPTION OF THE PLANT

The following is a detailed description of our new variety of Ilex made from observation of the original seedling growing in the ground in the vicinity of New Brunswick, N.J. and of nine-year-old plants growing in the ground at Adelphia, N.J.

Parentage: F₂ generation pistillate seedling of (Ilex×Meserveae)×Ilex pernyi.

Pollinator.—Plant may be pollinated by a staminate plant of either Ilex aquifolium, Ilex pernyi, Ilex rugosa, Ilex×aquipernyi, or Ilex×Meserveae with synchronous flowering period.

Plant growth characteristics: Plants have an upright, rather narrow conical habit with nine-year individuals growing to approximately 1.75 to 2.0 m in height and 1.0 m in width. Plants establish a dominant central trunk and branching is dense and self-compacting with very little or no pruning. Plants bear annual abundant crops of beautiful red berries which are attractively displayed among the semi-glossy dark green, spiny leaves which do not become lighter green when bearing a heavy crop of fruit as do the leaves of many varieties of Ilex.

Foliage:

Type.—Evergreen, coriaceous, semi-glossy. Leaves primarily ovate, slightly curved and keeled, spinose margin bearing an average of 11 small spines, slightly reflexed acute tip; and a very mildly rugose surface.

Size.—Leaf size varies with light intensity and fertility program where plants are growing. Size is generally in the range of 3-4.5 cm long and 2.0-2.6 cm wide.

Petiole.—Approximately 3-4 mm long and 1-2 mm wide, color is Yellow-Green Group 146C.

Color.—Mature levels, upper surface — closest to but slightly greener than Green Group 139A; under surface — Yellow-Green Group 146C. Leaves are semi-glossy.

Stems:

Color.—Tip 2.5-3 cm of new spring growth is Yellow-Green Group 144A and basal area is Yellow-Green Group 146B with stripes of a slight reddish tinge. One year old stems are Yellow-Green Group 146B; older branches and the central stem are Yellow-Green Group 146B with stripes of Greyed-Green Group 197C, and base of the tree trunk is Greyed-Green Group 197C.

Inflorescence and fruit:

Flowers.—Small on pedicels 2 to 4 mm long borne on 1 mm spurs in leaf axils of previous year's growth; regular calyx, corolla rotate, sepals 4-5, petals 4-5; petal length averages 3.6 mm, petal width averages 2.8 mm; petals of swollen flower buds exhibit a reddish blotch at tip ⅓ of dorsal surface and extending as a minute reddish stripe toward the petal base, the upper surface of the petals of an open flower are White Group 155A.

Fruit:

Type.—Drupe containing 4, occasionally 5, woody pyrenes.

Size.—Rounded, approximately 0.95 cm, with a black, 4-5 lobed stigmatic scar.

Color.—At maturity fruit are semi-glossy, Red Group 45A.

Environmental tolerance: Field grown plants of this new variety exhibit winter hardiness superior to most, if not all, hybrid varieties of hollies of erect, single-trunked tree habit that involve Ilex aquifolium and/or Ilex pernyi as a parent. No insect or disease damage has been observed on plants growing in the field. Asexual reproduction by cuttings and grafts of our new variety has been accomplished in the vicinity of New Brunswick, N.J. Propagules have shown that the distinctive combination of characteristic has been established and faithfully transmitted to successive generations.

What we claim is:

1. A new and distinct interspecific hybrid variety of Ilex named ‘Rutzan’ substantially as herein shown and described.
